(19) United States
(12) Patent Application Publication
(10) Pub. No.: US 2015/0307902 A1
(43) Pub. Date: Oct. 29, 2015

(54) RECYCLING OF ENZYMES FROM BIOREACTORS

(71) Applicant: Allan Otto Fog LIHME, Busan (KR)

(72) Inventor: Allan Otto Fog LIHME, Busan (KR)

(21) Appl. No.: 13/806,052

(22) PCT Filed: Jun. 24, 2011
(86) PCT No.: PCT/EP11/60667

§ 371 Date: Mar. 8, 2013

(30) Foreign Application Priority Data
Jun. 24, 2010 (GB) 10101644.1

(51) Int. Cl. C12P 7/10; C12P 7/16; C12P 7/64; C12P 7/04

(52) U.S. Cl. CPC C12P 7/10; C12P 7/04; C12P 7/16; C12P 7/649

(57) ABSTRACT

A method of conducting an enzymatic process in which the enzymes are recovered and reused in at least a second iteration of the process, wherein each iteration of the process comprises the steps of:
(a) providing a heterogeneous substrate solution, in which the substrate is fully soluble, partially soluble or insoluble;
(b) adding an enzyme or mixture of enzymes to the heterogeneous substrate solution; and
(c) allowing an enzymatic reaction of the substrate to proceed;
   wherein, after completion of step (c) for each iteration, the enzyme is recovered from the mixture resulting from step (c) according to the following steps:
(d) conducting a non-packed-bed adsorption process comprising contacting the reaction mixture with an adsorbent that adsorbs the enzyme in order to separate the enzyme from the reaction mixture resulting from step (c);
(e) optionally washing unbound material from the adsorbent; and
(f) desorbing the enzyme from the adsorbent;
   and further wherein the desorbed enzyme obtained in step (f) is used in step (b) of at least one subsequent iteration of the process.

The present invention relates to the recycling of enzymes from bioreactors using affinity capture with expanded bed adsorption (EBA) systems.

Biologically produced alcohols, most commonly ethanol, and less commonly propanol and butanol, are produced by the action of microorganisms and enzymes through the fermentation of sugars or starches (simplest procedure), or cellulose (which is more complex). Biobutanol (also called biogasoline) is often claimed to provide a direct replacement for gasoline, because it can be used directly in a gasoline engine (in a similar way to biodiesel in diesel engines).

Ethanol fuel is the most common biofuel worldwide, particularly in Brazil. Alcohol fuels are produced by fermentation of sugars derived from wheat, corn, sugar beets, sugar cane, molasses and any sugar or starch that alcoholic beverages can be made from (like potato and fruit waste, etc.). The ethanol production methods used are enzyme digestion (to release sugars from stored starches), fermentation of the sugars, distillation and drying. The potential quantity of ethanol that could be produced from cellulose is over an order of magnitude larger than that producible from corn. In contrast to the corn-to-ethanol conversion, the cellulose-to-ethanol route involves little or no contribution to the greenhouse effect and has a clearly positive net energy balance. As a result of such considerations, microorganisms that metabolize cellulose have gained prominence in recent years.

Lignocellulose is difficult to hydrolyze because (i) it is associated with hemicellulose, (ii) it is surrounded by a lignin seal which has a limited covalent association with hemicellulose, and (iii) much of it has a crystalline structure with a potential formation of six hydrogen bonds, four intramolecular and two intermolecular, giving it a highly ordered, tightly packed structure. Pretreatments aim at increasing the surface area of cellulose by (i) removing the lignin seal, (ii) solubilizing hemicellulose, (iii) disrupting crystallinity, and/or (iv) increasing pore volume. The value of a cellulase system that attacks crystalline cellulose lies in the observation that many of the pretreatments which increase surface area also increase crystallinity. These include dilute sulfuric acid, alkali, and ethylenediamine.

The rate-limiting step in the conversion of cellulose to fuels is its hydrolysis, especially the initial attack on the highly ordered, insoluble structure of crystalline cellulose, since the products of this attack are readily solubilized and converted to sugars. A great deal of effort has gone into the development of methods for conversion of cellulose to sugars. Most of this work has emphasized the biochemistry, genetics, and process development of pretreatment methods and enzymatic breakdown.

Biofuel production is hampered by poor production economy partially due to the costs of the enzymes used (cellulases, xylanases, lignases and others) for dissolution of the biomass and the transformation of polymeric carbohydrate structures into fermentable sugars. It is typically necessary to balance the amount of enzyme used in order to reach maximal yield of biofuel product against the costs associated with the use of high enzyme concentrations (i.e. higher product yields could be achieved by addition of more enzyme—but the cost of doing this is prohibitive).

The enzymes involved, namely, cellulases, xylanases, lignases and others, are of high value. However, it is not currently the practice to recover and reuse the enzymes used in this process.

The present inventors have recognised that it would be desirable to recycle the enzymes at the end of the digestion process so that they may be reused. This is because the use of enzyme recycling not only generally lowers the cost of enzymes but also enables higher biofuel product yields and improved profitability.

The biomass in the digestion process is of very varied origin and will be composed of soluble, partly soluble and insoluble materials. Only a proportion of the biomass will be converted into di or monosaccharides, leaving a mixed solution of soluble and insoluble materials. Extracting the digestive enzymes from such a mixture presents a difficult challenge as conventional separation techniques such as membrane filtration or packed bed chromatography are unable to deal with the large quantities of insoluble material present without fouling and flow blockage.

Further, in order that the enzymes may be recovered from the mixture remaining after the digestion process is complete without destroying the activity and/or usefulness of the enzymes, it would be necessary to select an extraction method that can be conducted under mild conditions that do not lead to enzyme denaturation or deactivation.

The present invention provides a method of conducting an enzymatic process in which the enzymes are recovered and reused in at least a second iteration of the process, wherein each iteration of the process comprises the steps of:

(a) providing a heterogeneous substrate solution, in which the substrate is fully soluble, partially soluble or insoluble;

(b) adding an enzyme or mixture of enzymes to the heterogeneous substrate solution; and (c) allowing an enzymatic reaction of the substrate to proceed;

wherein, after completion of step (c) for each iteration, the enzyme is recovered from the mixture resulting from step (c) according to the following steps:

(d) conducting a non-packed-bed adsorption process comprising contacting the reaction mixture with an adsorbent that adsorbs the enzyme in order to separate the enzyme from the reaction mixture resulting from step (c);

(e) optionally washing unbound material from the adsorbent; and (f) desorbing the enzyme from the adsorbent;

and further wherein the desorbed enzyme obtained in step (f) is used in step (b) of at least one subsequent iteration of the process.

Preferably, the process is conducted for at least two iterations, such as three, five or ten iterations.

Preferably, the recycled enzyme used in the second and subsequent iterations is supplemented where necessary with additional enzyme in order to maintain the desired level of enzyme activity in the heterogeneous substrate solution, as it is expected that over time recovery of the enzyme from the reaction mixture will not be complete, and that the activity of the recovered enzyme will decrease with use. The supplementary enzyme may suitably be previously used and recovered enzyme or may be “fresh”, ie previously unused, enzyme.

Suitably, the method may further include a step in which the desorbed enzyme is obtained from the adsorbent in step (f) in aqueous solution and the aqueous solution is subjected to ultrafiltration to provide a concentrated enzyme solution and water. This provides the further economic and environmental benefit that the water used to recover the enzyme may be recycled, for example for use in desorbing further enzyme from the adsorbent.

Suitably, the adsorption step (d) of the present invention may be conducted simultaneously with other processes conducted on the mixture resulting from step (c). For example, where the process forms part of a process of producing biofuel, the adsorption step (d) may be conducted while fermentation of the sugars produced in step (c) is also carried out.

Various methods of non-packed-bed chromatography are known and may be used in the present invention. Packed bed chromatographic methods are considered unsuitable due to the propensity of packed columns of adsorbent to block when a heterogeneous solution, ie one containing insoluble material, is passed through such a column. However, methods in which a tank of the reaction mixture is contacted with the adsorbent, adsorption of the enzyme is permitted to take place, and subsequently the adsorbent is separated from the tank, such as by filtration, may suitably be used, along with other similar methods known in the art. Preferably, however, step (d) of the invention is conducted by expanded bed chromatography. Expanded bed chromatography (EBA) is a successful method for carrying out an affinity separation step to capture specific materials from unclarified feedstocks. In this respect it is well suited to the capture and recycling of high value enzymes from biomass digestion processes. The material to be processed on an EBA column can be defined as a heterogeneous solution containing undigested material and soluble digestion products after completion of the digestion process, this is an aqueous solution comprising more than 0.1% v/v of insoluble matter.

Suitably, the step (d) is conducted as a batch process. Suitably, the step (d) may use an adsorbent comprising high density particles, low density particles or magnetic particles, or alternatively may use a membrane adsorption process.

The substrate for the enzymatic process may be: cellulose or cellulose-containing material, such as wood or straw; starch-based material derived from corn, wheat or algae; an oil containing material derived from oil seeds, rape seed, sunflower, jatropha, or algae; or an insoluble or partly soluble plant protein solution.

The heterogeneous solution or suspension containing biomass can also comprise an organic solvent or an ionic solvent. Where this is the case, the digestion step (c) must take place using enzymes active under these conditions. Similarly, the adsorbent chosen to adsorb the enzyme in step (d) must be selected to be stable in organic or ionic liquids.

Suitable enzymes for use in the process of the invention include cellulase, xylanase and lignase enzymes. These enzymes may be naturally occurring or may be engineered to have desired properties.

Preferably, the enzyme or mixture of enzymes are labelled to enhance the specificity and/or strength of binding of the enzyme to the adsorbent.

Preferably, the enzyme or enzymes used in the present invention are genetically or chemically modified in order that they can more efficiently be captured by a particular adsorbent. Suitable labelling methods and chemical groups include:

incorporating a His6 tag for interaction with immobilised metal chelates or other specific peptide sequences that can be recognised specifically, or which bind to immobilised moieties such as dyes, eg. Cibacron Blue;

labelling both the enzymes and the adsorbent with dyes and using a bridging molecule such as albumin to effect immobilisation;

labelling the enzyme with the dye, generally this can be on the end of a spacer molecule such as dextran or polyethylene glycol and the labelled enzyme can then bind to the albumin immobilised to the adsorbent;

binding specific sequences to the enzymes including DNA oligomers or analogues such as PNA or LNA that will bind to complementary sequences immobilised on the adsorbent;

modifying the enzymes with carbohydrate moieties or acetylation, succinylation, alkylation or reductive amination; and

affinity labelling of the enzymes such as by biotin, reactive dyes or boronic acids, chelating groups e.g. IDA, cyclodextrins, polyethylene glycols or dextrans with attached ligands.

A preferred method is to label the enzyme with a specific ligand, such as fluorescein, and to immobilise a ligand-specific antibody, such as a fluorescein-specific antibody, on the absorbent, with elution later by reducing the pH to reverse the antibody/ligand interaction. It is advantageous to use a low cost source of antibody, such as plant derived materials, or ‘plantibodies’.

An alternative approach is to modify the enzyme to substantially change the pI, without decreasing substantially the enzymatic activity, such that the pI differs from other proteins present in the digestion process. Thus under the pH conditions chosen for the chromatography capture step the modified enzyme will be bound by the adsorbent and there will be negligible binding of other proteins. Preferably the adsorbent will be an ion exchanger and elution of the bound enzyme will be effected by changing the pH of the elution buffer.

A further approach is to use as the adsorbent an immobilised substrate for the enzyme that binds to the active site with sufficiently high affinity to allow capture of the enzyme from the digestion liquid.

A still further approach is to label the enzyme with a ferromagnetic, paramagnetic or superparamagnetic substance such as dextran coated iron oxide nanoparticles or microparticles or other polymeric magnetic conjugates capable of being strongly bound to the enzyme.

Another approach is to use synthetic ligands on the adsorbent to bind directly to the enzyme in the digestion liquidor to bind to a domain inserted into the enzyme amino acid sequence specifically to interact with a ligand, or to bind to a moiety that has been introduced by chemical derivatisation. The bound enzyme can then be released from the adsorbent subsequently using a suitable change in conditions, such as pH or ionic strength.

In an EBA process the flow rate, the size of the particles and the density of the particles all have influence on the expansion of the fluid bed and it is important to control the degree of expansion in such a way to keep the particles inside the column. The degree of expansion may be determined as H/HO, where HO is the height of the bed in packed bed mode and H is the height of the bed in expanded mode. In a preferred embodiment of the present invention the degree of expansion H/HO is in the range of 1.0-20, such as 1.0-10, e.g. 1.0-6, such as 1.2-5, e.g. 1.5-4 such as 4-6, such as 3-5, e.g. 3-4 such as 4-6. In another preferred embodiment of the present invention the degree of expansion H/HO is at least 1.0, such as at least 1.5, e.g. at least 2, such as at least 2.5, e.g. at least 3, such as at least 3.5, e.g. at least 4, such as at least 4.5, e.g. at least 5, such as at least 5.5, e.g. at least 6, such as at least 10, e.g. at least 20. The density of the EBA adsorbent particle is found to be highly significant for the applicable flow rates in relation to the maximal degree of expansion of the adsorbent bed possible inside a typical EBA column (e.g. H/HO max 3-5) and must be at least 1.3 g/mL, more preferably at least 1.5 g/mL, still more preferably at least 1.8 g/mL, even more preferably at least 2.0 g/mL, most preferably at least 2.3 g/mL in order to enable a high productivity of the process. The density of an adsorbent particle is meant to describe the density of the adsorbent in its fully solvated (e.g. hydrated) state as opposed to the density of a dried adsorbent. In a preferred embodiment of the present invention the adsorbent particle has a mean particle diameter of at most 150 μm, particularly at most 120 μm, more particularly at most 100 μm, even more particularly at most 90 μm, even more particularly at most 80 μm, even more particularly at most 70 μm. Typically the adsorbent particle has a mean particle diameter in the range of 40-150 μm, such as 40-120 μm, e.g. 40-100, such as 40-75, e.g. 40-50 μm. In a combination of preferred embodiments, where the mean particle diameter is 120 μm or less, the particle density is at least 1.6 g/mL, more preferably at least 1.9 g/mL. When the mean particle diameter is less than 90 μm the density must be at least 1.8 g/mL or more preferably at least 2.0 g/mL. When the mean particle diameter is less than 75 μm the density must be at least 2.0 g/mL, more preferably at least 2.3 g/mL and most preferably at least 2.5 g/mL. The high density of the adsorbent particle is, to a great extent, achieved by inclusion of a certain proportion of a dense non-porous core materials, preferably having a density of at least 4.0 g/mL, such as at least 5.0, Typically, the non-porous core material has a density in the range of about 4.0-25 g/ml, such as about 4.0-20 g/ml, e.g. about 4.0-15 g/mL, such as 12-19 g/ml, e.g. 14-18 g/ml, such as about 6.0-15.0 g/mL, e.g. about 6.0-10 g/ml. The enzyme-containing mixture can be applied to the adsorbent column at a linear flow rate of at least 3 cm/min, such as at least 5 cm/min, e.g. at least 8 cm/min, such as at least 10 cm/min e.g. 20 cm/min. Typically the flow rate is selected in the range of 5-50 cm/min, such as in the range of 5-30 cm/min, e.g. in the range of 10-30 cm/min, such as in the range of 25-50 cm/min. Increased flow rates are possible to a great extent due to the small particle size of the adsorbent, thus the application of digested biomass and enzymes to the adsorbent column is with a linear flow rate of at least 200 cm/hour, such as at least 300 cm/hour, more preferably at least 400 cm/hour, such as at least 500 or 600 cm/hour, such as at least 900 cm/hour. In a combination of particularly preferred embodiments of the invention, where the applied linear flow rate during application of the digested biomass material is above 300 cm/hour, the preferred mean particle diameter is below 150 μm. Typically, in embodiments where the enzyme recovery process is performed at an applied linear flow rate of above 500 cm/min the mean particle diameter is below 120 μm, preferably below 90 μm. Typically, in embodiments where the enzyme recovery process is performed at an applied linear flow rate of above 600 cm/hour, the mean particle diameter is preferably below 85 μm, more preferably below 75 μm. The adsorbent particle used according to the invention is preferably at least partly permeable to the enzyme to be recovered in order to ensure a significant binding capacity in contrast to impermeable particles that can only bind the target molecule on its surface resulting in relatively low binding capacity. The adsorbent particle may be of an array of different structures, compositions and shapes. Thus, the adsorbent particles may be constituted by a number of chemically derivatised porous materials having the necessary density and binding capacity to operate at the given flow rates per se. The particles are either of the conglomerate type, as described in WO 10 92/00799, having at least two non-porous cores surrounded by a porous material, or of the pellicular type having a single non-porous core surrounded by a porous material. In the present context the term “conglomerate type” relates to a particle of a particulate material, which comprises beads of core material which may be of different types and sizes, held together by the polymeric base matrix, e.g. a core particle consisting of two or more high density particles held together by surrounding agarose (polymeric base matrix). In the present context the term “pellicular type” relates to a composite particle, wherein each particle consists of only one high density core material coated with a layer of the porous polymeric base matrix, e.g. a high density stainless steel bead coated with agarose. Accordingly the term “at least one high density non-porous core” relates to either a pellicular core, comprising a single high-density non-porous particle or it relates to a conglomerate core comprising more than one high density non-porous particle. The adsorbent particle, as stated, comprises a high density non-porous core with a porous material surrounding the core, and said porous material optionally comprising a ligand at its outer surface. In the present context the term “core” relates to the non-porous core particle or core particles present inside the adsorbent particle. The core particle or core particles may be randomly distributed within the porous material and is not limited to be located in the centre of the adsorbent particle. The non-porous core constitutes typically at most 50% of the total volume of the adsorbent particle, such as at most 40%, preferably at most 30%. Examples of suitable non-porous core materials are inorganic compounds, metals, heavy metals, elementary non-metals, metal oxides, non metal oxides, metal salts and metal alloys, etc. as long as the density criteria above are fulfilled. Examples of such core materials are metal silicates metal borosilicates; ceramics including titanium diboride, titanium carbide, zirconium diboride, zirconium carbide, tungsten carbide, silicon carbide, aluminum nitride, silicon nitride, titanium nitride, yttrium oxide, silicon metal powder, and molybdenum disilide; metal oxides and sulfides, including magnesium, aluminum, titanium, vanadium, chromium, zirconium, hafnium, manganese, iron, cobalt, nickel, copper and silver oxide; non-metal oxides; metal salts, including barium sulfate; metallic elements, including tungsten, zirconium, titanium, hafnium, vanadium, chromium, manganese, iron, cobalt, nickel, indium, copper, silver, gold, palladium, platinum, ruthenium, osmium, rhodium and iridium, and alloys of metallic elements, such as alloys formed between said metallic elements, e.g. stainless steel; crystalline and amorphous forms of carbon, including graphite, carbon black and charcoal. Preferred non-porous core materials are tungsten carbide, tungsten, steel and titanium beads such as stainless steel beads. The porous material is a polymeric base matrix used as a means for covering the core and, where necessary, keeping multiple (or a single) core materials together and as a means for binding the adsorbing ligand. The polymeric base matrix may be sought among certain types of natural or synthetic organic polymers, typically selected from i) natural and synthetic polysaccharides and other carbohydrate based polymers, including agar, alginate, carrageenan, guar gum, gum arabic, gum ghatti, gum tragacanth, karaya gum, locust bean gum, xanthan gum, agaroses, celluloses, pectins, mucins, dextrans, starches, heparins, chitosans, hydroxy starches, hydroxypropyl starches, carboxymethyl starches, hydroxyethyl celluloses, hydroxypropyl celluloses, and carboxymethyl celluloses; ii) synthetic organic polymers and monomers resulting in polymers, including acrylic polymers, polyamides, polyimides, polyesters, polyethers, polymeric vinyl compounds, polyalkenes, and substituted derivatives thereof, as well as copolymers comprising more than one such polymer functionality, and substituted derivatives thereof; and iii) mixture thereof. A preferred group of polymeric base matrices are polysaccharides such as agarose. From a productivity point of view it is important that the adsorbent is able to bind a high amount of the enzyme to be recycled per volume unit of the adsorbent. Thus we have found that it is preferable to use adsorbents having a polymeric phase (i.e. the permeable backbone where the ligand is positioned and whereto the actual adsorption is taking place) which constitutes at least 50% of the adsorbent particle volume, preferably at least 70%, more preferably at least 80% and most preferably at least 90% of the volume of the adsorbent particles. The investigators of the present invention have found that in order to ensure an efficient adsorption at high flow rates it is preferred to minimise the mean particle diameter of the adsorbent particle. The preferred shape of a single adsorbent particle is substantially spherical. The overall shape of the particles is, however, normally not extremely critical, thus, the particles can have other types of rounded shapes, e.g. ellipsoid, droplet and bean forms. However, for certain applications (e.g. when the particles are used in a fluidised bed set-up), it is preferred that at least 95% of the particles are substantially spherical.

Preparation of the particulate material according to the invention may be performed by various methods known per se (e.g. by conventional processes known for the person skilled in the art, see e.g. EP 0 538 350 B1 or WO 97/17132. For example, by block polymerization of monomers; suspension polymerization of monomers; block or suspension gelation of gel-forming materials, e.g. by heating and cooling (e.g. of agarose) or by addition of gelation “catalysts” (e.g. adding a suitable metal ion to alginates or carrageenans); block or suspension cross-linking of suitable soluble materials (e.g. cross linking of dextrans, celluloses, or starches or gelatines, or other organic polymers with e.g. epichlorohydrin or divinyl sulphone); formation of silica polymers by acidification of silica solutions (e.g. block or suspension solutions); mixed procedures e.g. polymerization and gelation; spraying procedures; and fluid bed coating of density controlling particles; cooling emulsions of density controlling particles suspended in polymeric base matrices in heated oil solvents; or by suspending density controlling particles and active substance in a suitable monomer or copolymer solution followed by polymerization. In a particularly suitable embodiment generally applicable for the preparation of the particulate material according to the invention, a particulate material comprising agarose as the polymeric base matrix and steel beads as the core material is obtained by heating a mixture of agarose in water (to about 95° C.), adding the steel beads to the mixture and transferring the mixture to a hot oil (e.g. vegetable oils), emulsifying the mixture by vigorous stirring (optionally by adding a conventional emulsifier) and cooling the mixture. This process can be carried out in a continuous manner, or by emulsion polymerisation in a continuous process, see WO2009071560. It will be appreciated by the person skilled in the art that the particle size (i.e. the amount of polymeric base matrix (here: agarose) which is incorporated in each particle can be adjusted by varying the speed of the mixer and the cooling process. Typically, following the primary production of a particle preparation the particle size distribution may be further defined by sieving and/or fluid bed elutriation.

A method of binding the enzyme to the porous matrix, such as polymer agarose, is to chemically derivatise with a low molecular weight ligand with affinity to the enzyme to be recycled. The ligand constitutes the adsorbing functionality of the adsorbent media or the polymeric backbone of the adsorbent particle has a binding functionality incorporated per se. Such affinity ligands may be linked to the base matrix by methods known to the person skilled in the art, e.g. as described in “Immobilized Affinity Ligand Techniques” by Hermanson et al., Academic Press, Inc., San Diego, 1992. The ligands may be attached to the solid phase material by any type of covalent bond known per se to be applicable for this purpose, either by a direct chemical reaction between the ligand and the solid phase material or by a preceding activation of the solid phase material or of the ligand with a suitable reagent known per se making it possible to link the matrix backbone and the ligand. Examples of such suitable activating reagents are epichlorohydrin, epibromohydrin, allyl-glycidylether; bis-epoxides such as butanedioldiglycidylether; halogen-substituted aliphatic compounds such as di-chloro-propanol, divinyl sulfone; carbonyldiimidazole; aldehydes such as glutaric dialdehyde; quinones; cyanogen bromide; periodates such as sodium-meta-periodate; carbodiimides; chloro-triazines such as cyanuric chloride; sulfonyl chlorides such as tosyl chlorides and tresyl chlorides; N-hydroxy succinimides; 2-fluoro-1-methylpyridinium toluene-4-sulfonates; oxazolones; maleimides; pyridyl disulfides; and hydrazides. Among these, the activating reagents leaving a spacer group different from a single bond, e.g. epichlorohydrin, epibromohydrin, allyl-glycidylether; bis-epoxides; halogen-substituted aliphatic compounds; divinyl sulfone; aldehydes; quinones; cyanogen bromide; chloro-triazines; oxazolones; maleimides; pyridyl disulfides; and hydrazides, are preferred.

Especially interesting activating reagents are believed to be epoxy-compounds such as epichlorohydrin, allyl-glycidylether and butanedioldiglycidylether.

In certain instances the activating reagent may even constitute a part of the functionality contributing to the binding of enzymes to the solid phase matrix. e.g. in cases where divinyl sulfone is used as the activating reagent. In other cases the activating reagent is released from the matrix during reaction of the functional group with the matrix. This is the case when carbodiimidazoles and carbodiimides are used.

The above mentioned possibilities make it relevant to define the presence of an optional spacer SP1 linking the matrix M and the ligand L. In the present context the spacer SP1 is to be considered as the part of the activating reagent which forms the link between the matrix and the ligand. Thus, the spacer SP1 corresponds to the activating reagents and the coupling reactions involved. In some cases, e.g. when using carbodiimides, the activating reagent forms an activated form of the matrix or of the ligand reagent. After coupling, no parts of the activating reagent are left between the ligand and the matrix, and, thus, SP1 is simply a single bond.

In other cases the spacer SP1 is an integral part of the functional group effecting the binding characteristics, i.e. the ligand, and this will be especially significant if the spacer SP1 comprises functionally active sites or substituents such as thiols, amines, acidic groups, sulfone groups, nitro groups, hydroxy groups, nitrile groups or other groups able to interact through hydrogen bonding, electrostatic bonding or repulsion, charge transfer or the like.

In still other cases the spacer SP1 may comprise an aromatic or heteroaromatic ring which plays a significant role for the binding characteristics of the solid phase matrix. This would for example be the case if quinones or chlorotriazines where used as activation agents for the solid phase matrix or the ligand.

Preferably, the spacer SP1 is a single bond or a biradical derived from an activating reagent selected from epichlorohydrin, allyl-glycidylether, bis-epoxides such as butanedioldiglycidylether, halogen-substituted aliphatic compounds such as 1,3-dichloropropan-2-ol, aldehydes such as glutaric dialdehyde, divinyl sulfone, quinones, cyanogen bromide, chloro-triazines such as cyanuric chloride, 2-fluoro-1-methylpyridinium toluene-4-sulfonates, maleimides, oxazolones, and hydrazides.

Preferably the spacer SP1 is selected from short chain aliphatic biradicals, e.g. of the formula —CH₂—CH(OH)—CH₂— (derived from epichlorohydrin), —(CH₂)₃—O—CH₂—CH(OH)—CH₂— (derived from allyl-glycidylether) or —CH₂—CH(OH)—CH₂—O—(CH₂)₄—O—CH₂—CH (OH)—CH₂— (derived from butanedioldiglycidylether; or a single bond. The ligand structure may also be aromatic or heteroaromatic, may cover a very wide spectrum of different structures optionally having one or more substituents on the aromatic or heteroaromatic ring(s) groups (radicals) of the following types as functional groups: benzoic acids such as 2-aminobenzoic acids, 3-aminobenzoic acids, 4-aminobenzoic acids, 2-mercaptobenzoic acids, 4-amino-2-chlorobenzoic acid, 2-amino-5-chlorobenzoic acid, 2-amino-4-chlorobenzoic acid, 4-aminosalicylic acids, 5-aminosalicylic acids, 3,4-diaminobenzoic acids, 3,5-diaminobenzoic acid, 5-aminoisophthalic acid, 4-aminophthalic acid; cinnamic acids such as hydroxycinnamic acids; nicotinic acids such as 2-mercaptonicotinic acids; naphthoic acids such as 2-hydroxy-1-naphthoic acid; quinolines such as 2-mercaptoquinoline; tetrazolacetic acids such as 5-mercapto-1-tetrazolacetic acid; thiadiazols such as 2-mercapto-5-methyl-1,3,4-thiadiazol; benzimidazols such as 2-amino-benzimidazol, 2-mercaptobenzimidazol, and 2-mercapto-5-nitro-benzimidazol; benzothiazols such as 2-aminobenzothiazol, 2-amino-6-nitrobenzothiazol, 2-mercaptobenzothiazol and 2-mercapto-6-ethoxybenzothiazol;

benzoxazols such as 2-mercaptobenzoxazol; thiophenols such as thiophenol and 2-aminothiophenol; 2-(4-aminophenylthio)acetic acid; aromatic or heteroaromatic sulfonic acids and phosphonic acids, such as 1-amino-2-naphthol-4-sulfonic acid and phenols such as 2-amino-4-nitrophenol.

The ligand may have further substituents of the following formula -SP2-ACID wherein SP2 designates an optional second spacer and ACID designates an acidic group. In the present context the term “acidic group” is intended to mean groups having a pKa-value of less than about 6.0, such as a carboxylic acid group (—COOH), sulfonic acid group (—SO₂OH), sulfinic acid group (—S(O)OH), phosphinic acid group (—PH(O)(OH)), phosphonic acid monoester groups (—P(O)(OH)(OR)), and phosphonic acids group (—P(O)(OH)₂. The pKa-value of the acidic group should preferably be in the range of 1.0 to 6.0. The acidic group is preferably selected from carboxylic acid, sulfonic acid, and phosphonic acid. The group SP2 is selected from C₁₋₁₂-alkyl, C₁₋₆-alkylene, and C₂₋₆-alkenylene, or SP2 designates a single bond. Examples of relevant biradicals are methylene, ethylene, propylene, propenylene, isopropylene, and cyclohexylene. Preferably, SP2 designates methylene, ethylene, or a single bond. In the present context, the term “C₁₋₁₂-alkyl” is intended to mean alkyl groups with 1-12 carbon atoms which may be straight or branched or cyclic such as methyl, ethyl, propyl, isopropyl, butyl, isobutyl, tert-butyl, pentyl, hexyl, octyl, dodecyl, cyclopentyl, cyclohexyl, decalinyl, etc.

C₁₋₁₂-alkyl may be substituted with one or more, preferably 1-3, groups selected from carboxy; protected carboxy such as a carboxy ester, e.g. C₁₋₆-alkoxycarbonyl; aminocarbonyl; mono- and di(C₁₋₆-alkyl)-aminocarbonyl; amino-C₁₋₆-alkyl-aminocarbonyl; mono- and di(C₁₋₆-alkyl)amino-C₁₋₆-alkyl-aminocarbonyl; amino; mono- and di(C₁₋₆-alkyl)amino; (C₁₋₆-alkylcarbonylamino; hydroxy: protected hydroxy such as acyloxy, e.g. C₁₋₆-alkanoyloxy; sulfono; C₁₋₆-alkylsulfonyloxy; nitro; phenyl; phenyl; C₁₋₆-alkyl; halogen; nitrilo; and mercapto. Examples of substituted C₁₋₁₂-alkyl groups are carboxy-C₁₋₁₂-alkyl (e.g. carboxymethyl and carboxyethyl), protected carboxy-C₁₋₆-alkyl (e.g. C₁₋₁₂-alkyl such as esterified carboxy-C₁₋₆-alkyl (e.g. C₁₋₆-alkoxy-carbonyl-C₁₋₁₂-alkyl such as methoxycarbonylmethyl, ethoxycarbonylmethyl, and methoxycarbonylethyl), aminocarbonyl-C₁₋₁₂-alkyl (e.g. aminocarbonylethyl, aminocarbonylethyl and aminocarbonylpropyl), C₁₋₆-alkylaminocarbonyl-C₁₋₁₂-alkyl (e.g. methylaminocarbonylmethyl and ethylaminocarbonylmethyl), amino-C₁₋₆-alkyl-aminocarbonyl-C₁₋₁₂-alkyl (e.g. aminomethylaminocarbonylmethyl and aminoethylaminocarbonylmethyl), mono- or di(C₁₋₁₂-alkyl)amino-C₁₋₆-alkylaminocarbonyl-C₁₋₁₂-alkyl (e.g. dimethylaminomethylaminocarbonylmethyl and dimethylaminoethylaminocarbonylmethyl), mono- or di(C₁₋₆-alkyl)amino-C₁₋₁₂-alkyl (e.g. di-methylaminomethyl and dimethylaminoethyl), hydroxy-C₁₋₁₂-alkyl (e.g. hydroxymethyl and hydroxyethyl), protected hydroxy-C₁₋₁₂-alkyl such as acyloxy-C₁₋₁₂-alkyl (e.g. C₁₋₆-alkanoyloxy-C₁₋₁₂-alkyl such as acetyloxyethyl, acetyloxypropyl, acetyloxybutyl, acetyloxypentyl, propionyloxymethyl, butyryloxymethyl, and hexanoyloxymethyl).

In the present context, the term “C₂₋₁₂-alkenyl” is intended to mean mono-, di- or polyunsaturated alkyl groups with 2-12 carbon atoms which may be straight or branched or cyclic in which the double bond(s) may be present anywhere in the chain or the ring(s), for example vinyl, 1-propenyl, 2-propenyl, hexenyl, decenyl, 1,3-heptadienyl, cyclohexenyl etc. Some of the substituents exist both in a cis and a trans configuration. The scope of this invention comprises both the cis and trans forms.

In the present context, the term “C₂₋₁₂-alkynyl” is intended to mean a straight or branched alkyl group with 2-12 carbon atoms and incorporating one or more triple bond(s), e.g. ethynyl, 1-propynyl, 2-propynyl, 2-butynyl, 1,6-heptadiynyl, etc. The ligand structures should not be bound by any specific theory, however, it is envisaged that the special electronic configuration of the aromatic or heteroaromatic moiety in combination with one or more heteroatoms, which may be located in the heteroaromatic ring system or as a substituent thereon, is involved in the specific binding of enzymes.

### BRIEF DESCRIPTION OF THE FIGURES

FIG. 1 shows a schematic representation of a process according to the invention.

FIG. 2 shows schematically an enzyme recycling system including expanded bed adsorption and membrane filtration

FIG. 3 shows schematically a system for recovering enzyme during fermentation

FIG. 4 shows schematically a combined protein isolation and enzyme recycling plant

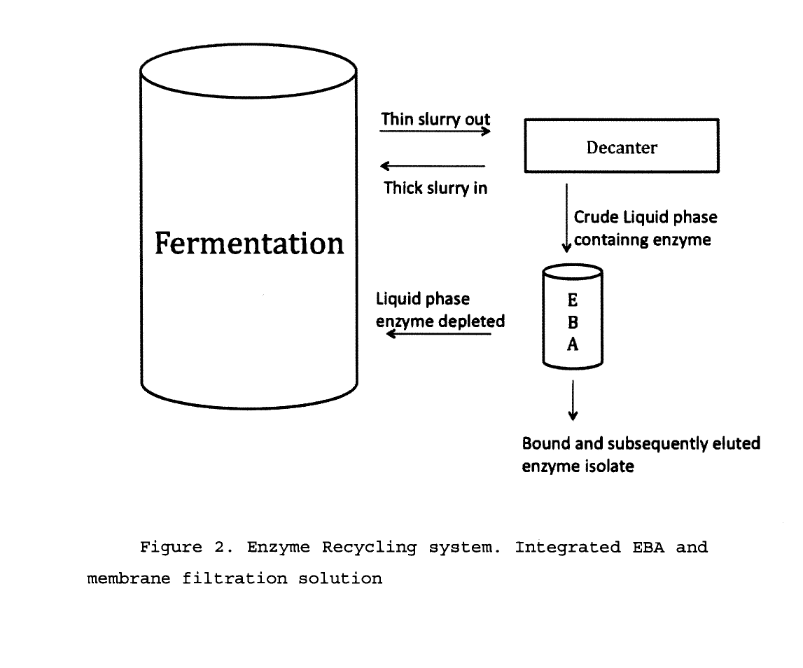

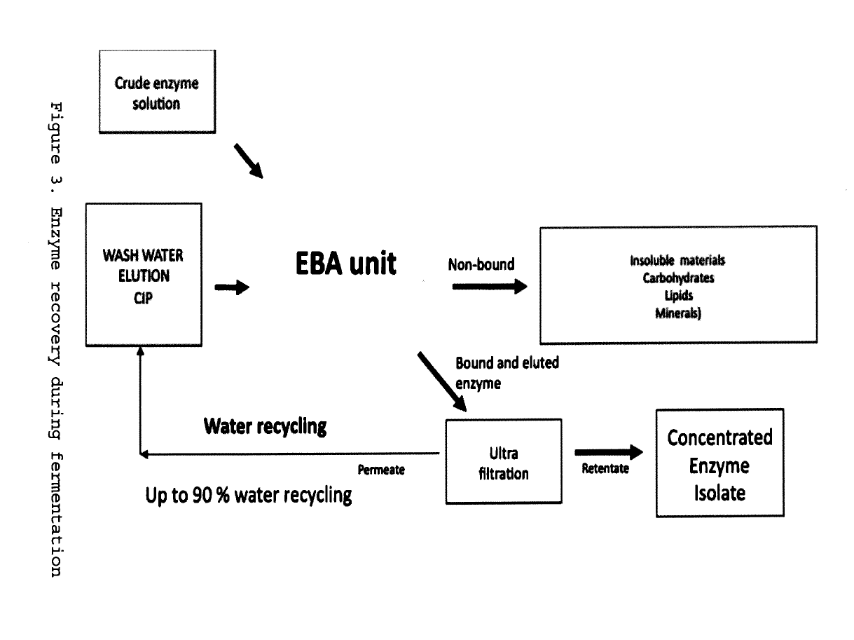

An integrated EBA enzyme recovery and membrane filtration process can be implemented where ultrafiltration is used as a water recovery step to improve the process economics, see FIG. 2. In this instance the crude enzyme solution can be taken from any stage in the bioethanol process. The EBA-based enzyme recycling process can also be used specifically at the biomass fermentation stage post pre-treatment, where enzyme recovery can take place alongside the yeast-based or bacterial-based fermenter, thus the enzyme recycling can be part of a simultaneous saccharification and fermentation (SSF) process, see FIG. 3.

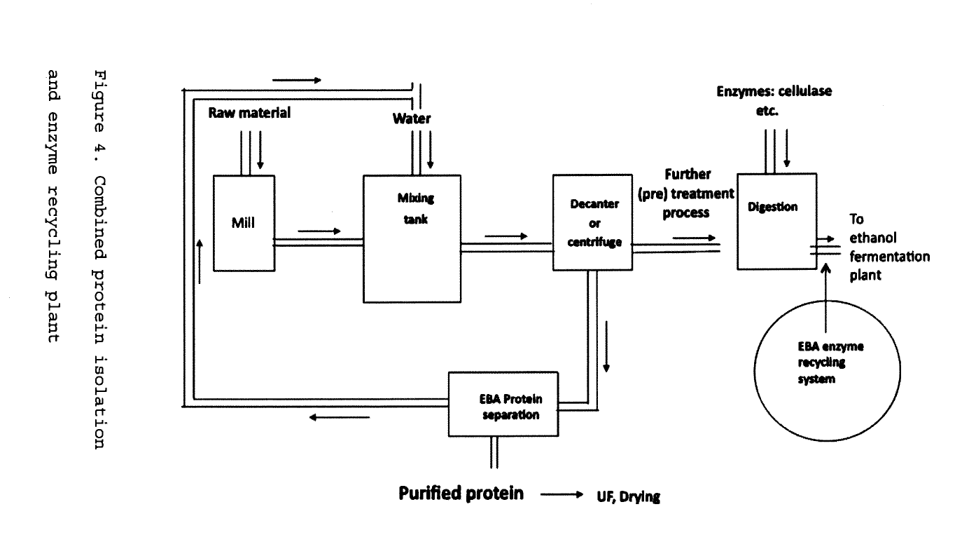

A combined soluble protein purification from biomass, by EBA adsorption, followed by a further, optional, pre-treatment step to prepare the sample for cellulose digestion and then an EBA-based enzyme recycling step is illustrated in FIG. 4. Note, in this combined process two EBA adsorption steps are used, the first removes commercially valuable soluble proteins from the dispersed biomass that would otherwise be destroyed by further processing, whilst the second recycles the digestion enzymes prior to fermentation.

### EXAMPLE

Fluorescein-affinity labelling of enzymes and recycling via immuno-adsorption to immobilised anti-fluorescein antibodies.

Preparation of Sheep-Anti Fluorescein Antibodies

Purified bovine serum albumin (Sigma cat. no.: A4503) is labelled with fluorescein isothiocyanate (Sigma Cat. no.: F4274) following the guidelines given in the Product Description to result in a fluorescein-BSA conjugate having a fluorescein/protein ratio of approx. 1 as determined by measurement of the conjugate absorbance at 280 nm and 495 nm respectively.

The fluorescein-BSA conjugate is then used for immunization of sheep following the guidelines given in

“Antibodies Volume I—A practical approach”, Chapter 2, pp 19-78, edited by D Catty, IRL Press Ltd, 1988. Following several months of repeated immunizations the sheep are bleed to produce a high titer anti-fluorescein sheep serum.

Divinyl Sulfone Activation of High Density Beads.

High density beads comprising agarose and tungsten carbide are prepared essentially as described in WO2009071560A1 to obtain highly regular beads comprising 4% agarose and having a density of approx. 2.6 g/ml and a mean particle size of approx. 150 micron.

Approximately 1400 ml of a 1:1 suspension of the high density agarose beads in water is washed with demineralised water on a sintered glass funnel followed by suction draining for one minute. 700 ml of wet, but drained, agarose beads are combined with 450 ml 0.5 M potassium phosphate buffer pH 11.5. 35 ml divinyl sulfone (Sigma-Aldrich Cat. no.: V 3700) is added and the resulting suspension is paddle stirred at room temperature for 2 hours. The matrix is then transferred to a sintered glass funnel and washed with 20 litres of water, 5 litres of 30% ethanol in water and finally 5 litres of water. The resulting activated matrix has a content of approx. 20 μmol active vinyl groups per ml suction drained beads.

Immobilization of Anti-Fluorescein Antibodies

500 ml divinyl sulfone activated high density agarose beads is combined with 1500 ml sheep-anti fluorescein serum as prepared above. To this suspension is added 110 g polyethylene glycol under gentle stirring with a paddle stirrer. The pH of the suspension is adjusted to pH 9.0 by the gradual addition of 1 M sodium hydroxide. The suspension is stirred at room temperature for 18 hours during which time preferentially the immunoglobulin (antibodies) in the sheep serum will be covalently immobilized to the high density beads through reaction with the vinyl sulfone groups hereon. Following coupling the beads are washed on a sintered glass filter with 5 L 0.5 M sodium chloride followed by 5 L 0.1 M potassium phosphate pH 11.0 followed by 5 L 0.5 M sodium chloride+0.1 M potassium phosphate pH 7.0.

Fluorescein Labelling of Cellulase

50 ml Celluclast 1.5L (Sigma Cat. No.: C2730), an acidic cellulase, is diafiltrated against 5 times 100 ml water using a polysulfone ultrafiltration membrane (GE Healthcare), 5000 Daltons molecular weight cut-off hollow fibre module. This procedure eliminates any low molecular weight substances that might interfere with the subsequent reaction with fluorescein isothiocyanate.

Following diafiltration the enzyme preparation is adjusted to a final protein concentration of 10 mg/ml (using the Coomassie Blue Reagent for determining protein concentration from BioRad Laboratories™, Richmond, Calif. and bovine serum albumin as the protein standard) and the pH is adjusted to pH 8.5. Immediately hereafter is added a solution of fluorescein isothiocyanate (2 mg/ml in dimethyl sulfoxide) in an amount corresponding to 100 microlitre per ml enzyme solution and the solution is reacted for 4 hours at room temperature. Following reaction the pH is adjusted to pH 7.0 with 1 M phosphoric acid surplus, un-reacted fluorescein isothiocyanate is removed from the enzyme solution by diafiltration as above, however, using 20 mM potassium phosphate pH 7.0 as the diafiltration buffer instead of water.

Substrate Preparation and Enzymatic Digestion Treatment

Dry corn stover is micronized by milling and pretreated with 1.4 wt % sulphuric acid at 165° C. and 107 psi for 8 minutes in a ratio of one part of corn stover to 3 parts of sulphuric acid. The insoluble material is then washed with 20 parts demineralised water on a sintered glass filter and drained for excess water by suction. The dry matter content is approx. 25%.

A suspension is prepared to reach 10% w/v of the pretreated corn stover in 50 mM sodium acetate pH 5.0 and SOFTANOL™ 90 (INEOS Oxide, Zwijndrecht, Belgium) is added to a final concentration of 1% v/v.

Fluorescein labelled Celluclast 1.5 L is added to reach an enzyme concentration of 10 mg/g pretreated corn stover and the suspended digestion reaction mixture is heated to 50 degrees Celsius under gentle stirring for 24 hours. Formation of reducing sugars is followed using the p-hydroxybenzoic acid hydrazide assay (Lever M., 1973, Colorimetric and fluorometric carbohydrate determination with p-hydroxybenzoic acid hydrazide, Biochemical Medicine 7: 274-281).

Following enzyme treatment, the heterogeneous reaction mixture is adjusted to pH 7 with 1 M sodium hydroxide and the enzyme is captured and recycled from the suspension using expanded bed adsorption as described below.

Enzyme Recycling Using Expanded Bed Adsorption and Elution into New Substrate Batch

The recycling procedure is performed in an expanded bed adsorption column, FastLine® 20, i.d.=2 cm from UpFront Chromatography A/S, Copenhagen, Denmark.

Following the general instructions to the Fastline 20 column, the column is initially packed with 50 cm of anti-fluorescein adsorbent (157 ml), as prepared above, and equilibrated prior to use by washing with 50 mM potassium phosphate pH 7.0 at 25° C. at an upwards linear flow rate of 10 cm/min provided by a peristaltic pump.

The enzyme treated corn stover at pH 7.0 is subsequently pumped through the expanded bed column at a linear flow rate of 10 cm/min, whereby the fluorescein labelled cellulase enzyme is selectively bound to the adsorbent via the immobilised anti-fluorescein sheep antibodies. Centrifuged samples (10000 rpm, 5 minutes) regularly withdrawn from the run-through from the expanded bed column are measured for the absorbance at 495 nm, which is an absorption maximum for fluorescein, thereby estimating the amount of non-bound fluorescein labelled protein passing the column. Loading of the column with enzyme treated corn stover is continued until the absorbance at 495 nm in the run-through samples reaches approx 20% of the absorbance of the centrifuged enzyme treated corn stover suspension prior to loading on the expanded bed column. The column is then washed with 50 mM phosphate buffer pH 7.0 to remove the remaining corn stover suspension.

The bound fluorescein labelled cellulase is then released and recycled into a new batch of pretreated, drained corn stover by washing the expanded bed column with 0.1 M sodium citrate buffer pH 3.0, until essentially all the bound fluorescein labelled protein is released, followed by final dry matter adjustment and pH adjustment to reach 10% corn stover dry matter and pH 5.0 for a new digestion process to begin. Any loss of enzyme activity throughout the prior digestion and recycling process is balanced by addition of additional fresh fluorescein labelled enzyme.

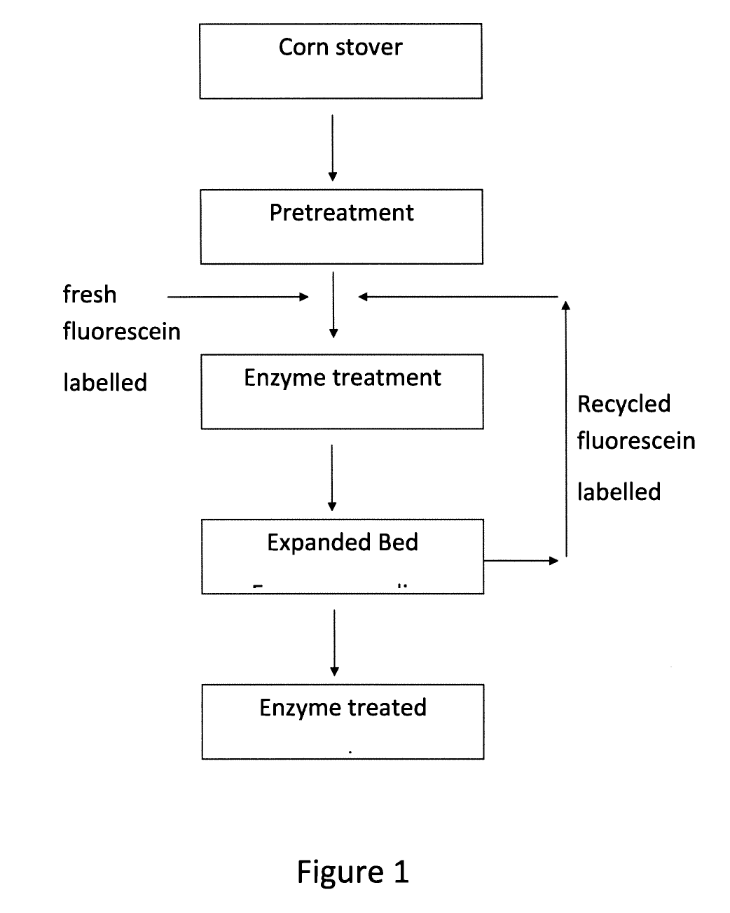

FIG. 1 illustrates the recycling principle as described in example 1.

1. A method of conducting an enzymatic process in which the enzymes are recovered and reused in at least a second iteration of the process, wherein each iteration of the process comprises the steps of:
(a) providing a heterogeneous substrate solution, in which the substrate is fully soluble, partially soluble or insoluble;
(b) adding an enzyme or mixture of enzymes to the heterogeneous substrate solution; and
(c) allowing an enzymatic reaction of the substrate to proceed;
wherein, after completion of step (c) for each iteration, the enzyme is recovered from the mixture resulting from step (c) according to the following steps:
(d) conducting a non-packed-bed adsorption process comprising contacting the reaction mixture with an adsorbent that adsorbs the enzyme in order to separate the enzyme from the reaction mixture resulting from step (c);
(e) optionally washing unbound material from the adsorbent; and
(f) desorbing the enzyme from the adsorbent;
and further wherein the desorbed enzyme obtained in step (f) is used in step (b) of at least one subsequent iteration of the process.

2. The recycling process as described in claim 1 repeated for two or more times with addition of further enzyme as required to replace losses and inactivation of enzyme activity

3. The process according to claim 1 wherein the non-packed bed adsorption process is an Expanded Bed adsorption process

4. A process according to claim 1 wherein the non-packed bed adsorption process is a batch adsorption process using high density, low density or magnetic adsorbent particles or a membrane adsorption process.

5. A process according to claim 1 wherein the enzyme or mixture of enzymes are labelled to enhance the specificity and/or strength of binding to the adsorbent.

6. A process according to claim 5 wherein the labelling of the enzyme or mixture of enzymes is performed by genetic modification.

7. A process according to claim 5 wherein the labelling of the enzyme or mixture of enzymes is performed by chemical modification of the enzyme(s).

8. A process according to claim 7 wherein the chemical modification comprises dyes, DNA oligomers or analogues such as PNA or LNA, carbohydrate moieties or acetylation, succinylation, alkylation, reductive amination, biotinylation, boronic acids, chelating groups e.g. IDA, cyclodextrins, polyethylene glycols or dextrans with attached ligands.

9. A process according to claim 1 wherein the substrate is cellulose or cellulose containing materials or starch or starch containing materials or oil containing materials or insoluble or partly insoluble plant protein solution.
